(19) United States
(12) Patent Application Publication
(10) Pub. No.: US 2009/0175925 A1
(43) Pub. Date: Jul. 9, 2009

(54) PASSIVE TIME RELEASED SOLUTE TREATMENT

(76) Inventor: Charles Nelson, Santa Rosa, CA (US)

Correspondence Address: LEVINE BAGADE HAN LLP, 2400 GENG ROAD, SUITE 120, PALO ALTO, CA, 94303, US

(21) Appl. No.: 12/190,496

(22) Filed: Aug. 12, 2008

Related U.S. Application Data
(63) Continuation of application No. PCT/US2007/062037, filed on Feb. 13, 2007.
(60) Provisional application No. 60/773,189, filed on Feb. 13, 2006.

(51) Int. Cl. A61C 19/06; A61K 31/197; A61K 31/56; B32B 3/26; A61J 7/00

(52) U.S. Cl. 424/435; 514/565; 514/169; 428/402; 604/77

(57) ABSTRACT

Devices and methods for delivery of a solute to increase blood sugar levels between certain hours in a form that allows for gradual delivery. One variation includes devices and methods for the timed release of a solute of carbohydrate, typically dextrose.

### CROSS-REFERENCE TO RELATED APPLICATIONS

This is a continuation of International PCT Application No. PCT/US2007/062037 filed Feb. 13, 2007 which is a non-provisional of U.S. Provisional Application No. 60/773,189 filed Feb. 13, 2006; the entireties of both applications are incorporated by reference herein.

### FIELD OF INVENTION

A need exist to provide a solute in a convenient form such that the solute may be delivered over a certain period of time. In one variation, the inventive devices and methods permit delivery of a solute to increase blood sugar levels between certain hours (e.g., 2:00 and 4:00 AM) in a more convenient, more reliable form than exists today. One variation of the invention includes devices and methods for the timed release of a solute of carbohydrate, typically dextrose. This invention disclosure describes a device that provides a timed release of dextrose. The design of this device increases the blood glucose level by 0 to 300 mg/dl, typically 30 to 90 mg/dl. Variations of the invention can increase the blood glucose level by delivering a single amount at a given time, gradually over a delayed amount of time, or a combination thereof (a large dosage followed by a gradual dosage). In one embodiment, the dosage increases the blood glucose level four hours after application of the device. As noted herein, one variation of the invention is a dental appliance that may be temporarily affixed to the roof of the mouth of the user.

### BACKGROUND OF THE INVENTION

Hypoglycemia is an adverse health condition where the blood sugar level falls to an undesirable level, typically below 60 milligrams per deciliter, mg/dl, or 3.3 millimoles per liter, mM. A further decrease in the concentration of sugar in the blood may result in death, cause brain damage, coma, or loss of consciousness. Typically, such extreme situations occur when the blood sugar level falls below 20 mg/dl.

There are several causes leading to hypoglycemia. These include exercise without proper pre or post exertion nutrition, excessive alcohol consumption and dehydration. However, the most prevalent cause of hypoglycemia is a disease state known as diabetes. A 2002 estimate shows that 170 million people suffer from diabetes worldwide. The estimated associated cost to society is over $134 billion (USD) per year. Annually, 2.9 million die due to complications directly caused by the diabetes. Of these deaths, 6%, or 174,000, are attributed to hypoglycemia. Similar estimates show that of these 174,000 deaths in the world, 15,000 die of hypoglycemia in the U.S. Acute morbidity resulting from nocturnal hypoglycemia presents a feared and significant change to their lives and the lives of their families. This disruption is especially concerning for children with diabetes.

People with diabetes (“PWD”) are categorized in many ways. These categories include gestational diabetes, Type I diabetes, Type II diabetes, pre-diabetes, and insulin dependent diabetes. Estimates typically show that 10% of the PWD are Type I, and an additional 21% of the total population of PWD's are insulin dependent. Therefore, up to 31% of the PWD's take insulin and 30% to 60% suffer severe hypoglycemic events. PWD, that are insulin dependent, have a greater incidence of hypoglycemia due to excessive dosage of insulin and low blood sugar. In the United States of America, there is an estimated 18 million PWD of which 5.6 million are insulin dependent.

Currently, monitoring and diet are methods for preventing hypoglycemia for PWD. Monitoring during waking hours is an effective method to adjust insulin and carbohydrate intake. However, at night a significant risk exists for severe hypoglycemia for insulin dependent PWD. If an individual with diabetes uses a pump, in some cases, the parent or individual will decrease their basal rate and allow increased blood sugar if the PWD has a propensity for nocturnal hypoglycemia. In the early hours of the morning a rebound effect, or Symoigi effect, can occur causing the blood sugar to rise to dangerous levels, over 250 mg/dl. Another method to prevent nighttime hypoglycemia is to eat a source of carbohydrates. At least three products are marketed to PWD as meal supplements. These are Glucerna from Abbott Laboratories, Extend Bar from Clinical Products, Ltd., and NiteBites from Valeant Pharmaceutical International. These products contain a mixture of protein, fat, polysaccharides, and dextrose. Except for Glucerna, the mixture of these food constituents provides immediate and delayed breakdown and adsorption of glucose into the blood.

However, the products above have several deficiencies. These products have poor taste and children with diabetes often resist eating these products prior to bedtime. The mixture of fat and polysaccharides, starches, cause weight gain and unwanted digestive side effects. The “science” of the conversion from of the product to blood sugar varies and is unpredictable. The Glucerna product provides a 50 mg/dl increase in blood sugar 60 minutes after ingesting the bar or fluid. This increase in blood sugar decays after 120 minutes and is eliminated after 24 hours. If the PWD takes the bar or shake at bed time, 9:00 to 10:00 PM, the increase in blood sugar is gone before 2:00 AM. Typically, parents of children with diabetes worry most about hypoglycemia between the hours of 2:00 and 4:00 AM. Therefore, PWD that rely upon a Glucerna-type product are still at risk of severe hypoglycemia. The Extend Bar advertises a 9 hours of slowly absorbed carbohydrates. This product utilizes uncooked corn starch which has the drawbacks mentioned earlier. Nitebites product was similar in performance to the Extend Bar.

### BRIEF SUMMARY OF THE INVENTION

The devices described herein include an oral appliance for retention within a mouth and effectuating a gradual increase in blood sugar levels, where the appliance includes a main body having a carrier surface and a engaging surface, where the engaging surface permits removable nesting and retaining of the main body within the mouth, and an active solute material located on the carrier surface. The active solute material consists of a solute, such as dextrose. The active solute material may be coated with a timed release coating, and other coatings, such as a pH active coating. The device can also include a combination of such coatings. The pH active coating dissolves at specific levels or sub-ranges of pH levels. The pH active layer or coating can be chosen to dissolve from pH levels above zero and up to 14. The active solute material releases the solute, in the case of dextrose, thereby increasing the blood sugar levels upon absorption through a digestive process. The active solute material may also contain or be combined with a time delayed release agent or pH active agent.

Depending on the application, the specific pH sub-range could be a below 4. Likewise, devices may have pH sub-ranges such as 1 to 4, or 8 to 14 or 5 to 6, etc. Alternatively, different layers of the device could have different pH sub-ranges. That is to say the pH activated layer dissolves as a function of the pH of the surrounding fluid. Furthermore, the pH activated layer can dissolve in surrounding fluids of a specific pH

The oral appliances may further comprise an inert timed release agent being dissolvable in saliva and covering the active solute material. Therefore, the break down of the inert time release agent layer causes release of the active solute material into the mouth.

The active solute material (e.g., dextrose) and/or the timed release agent can be coated with a pH activated layer that is insoluble in the pH of the saliva, but soluble in the pH of the stomach. Therefore, the active solute material is not released in the mouth. This embodiment prevents the active solute material from interacting with the teeth and causing tooth decay.

A variation of the appliance includes a main body having a surface for nesting and retaining within the mouth, and a layer of soluble material located on the main body, where the soluble material comprises an active solute adapted to increase blood sugar levels upon absorption through a digestive process and an inert timed release agent material, where a ratio of the active solute to inert timed release agent material varies along a depth of the layer.

Yet another variation of the dental appliance includes a device that causes a gradual release of an active solute material for purposes other than increasing the blood sugar level of the user. Such oral appliances may include a main body having a surface for nesting and retaining within the mouth, and a layer of soluble material located on the main body, where the soluble material comprises an active solute material adapted to effectuate a change in a body through a digestive process and an inert time release agent material, where a ratio of the active solute material to inert time release agent material varies along a depth of the layer.

In another variation of the invention, the concepts described herein may be applied to a time release agent substance that performs the same or similar function as those described herein. For example, another embodiment of the invention may include a particle or micro-particle. This particle may be made from a variety of inert, digestively inert, biocompatible materials for ingestion that include but are not limited to silicone rubber, fluorinated hydrocarbons, gelatin particles, polymers of styrene, latex, fumed silica, silica, and inert waxes of light hydrocarbons. Microparticles containing fluids are also envisioned. The structure, specifically the porosity, pore size, and pore size distribution can affect the release of the active agent, and in the case or dextrose, cause gradual increase in blood sugar levels in an individual. The particle may comprise a porous structure comprising an inert time release agent material, where the inert time release agent material is gradually dissolvable in digestive fluids or saliva, and an active solute material located within the porous structure and adapted to increase blood sugar levels upon absorption through a digestive process, such that the gradual dissolving of the porous structure timely releases the active solute material.

The micro-particle structure allows specific and modulated behavior of the dissolution in the body. The micro-particle can first be “loaded” with the active solute material, such as dextrose. The dextrose can then be coated with a layer that is soluble on at specific levels of pH or in the presence of specific enzymes or other bio-chemical or chemical agents. By this means, in one embodiment the micro-particle is coated with dextrose and then a layer of material that is soluble in only low pH, such as the pH of stomach acid, which is pH 4. A time delayed coating, such as hydroxymethylcellulose can then be applied over the pH sensitive coating. The hydroxymethylcellulose swells and dissolves after 2-4 hours. The particle then releases from the body of the carrier or dental appliance and is swallowed. The pH activated coating then dissolves in the stomach and the dextrose is released to be carried into the blood stream.

In another embodiment, the pH layer and the time delayed layer can be reversed. In this embodiment, the microparticle is coated with the solute, such as dextrose, the time delay coating is then applied so that the release of dextrose is delayed for a finite amount of time, such as 2 to 4 hours. Then the pH active layer is added over the time delayed coating. In this embodiment, the device may be in the form of an edible food, drink, or oral/dental appliance. The particle is swept immediately upon placement in the mouth into the stomach. The pH sensitive layer dissolves. Then the time release coating dissolves thereby releasing the dextrose into the stomach and eventually into the blood stream.

The main body of the device may be as described herein. However, it may be of any form that functions to nest within the mouth as a similar dental appliance.

The solutes/active layers described herein should be chosen to dissolve in a fluid, examples include dextrose, other monosaccharides, polysaccharides, starch, fat, protein, therapeutic agents (i.e. pharmacological agents), performance enhancement agents (creatine, steroids, etc).

The inert time release agent material or active solutes may dissolve between 0 and 72 hours. However, anywhere from 2-6 hours may be more desirable to accommodate a sleep schedule.

The body members and protrusions (where applicable) may be fabricated from a silicone rubber, a polymethylmethacrylate (PMMA), a bio-compatible metal such as stainless steel, titanium, or nickel titanium alloys or any biocompatible, metal, ceramic, rubber, gelatin, plastic or polymer suitable for placement in the mouth. The general classes of soluble and insoluble hydrogels, sodium algenates, other salts of alginate, strontium salts, strontium salts in hydrogels and gelatin are envisioned as body constituents or materials that encapsulate the active solute material.

The devices described herein can be sterilized by such standard processes as exposure to gamma, e-beam, ETO, steam, dry heat, or ultraviolet radiation.

The devices may also incorporate flavors, stabilizers, antioxidants, preservative, and food colorings.

It is important to note that, where possible, combinations of aspects of the various embodiments or the combination of the actual embodiments themselves are intended to be within the scope of this disclosure.

### BRIEF DESCRIPTION OF THE SEVERAL VIEWS OF THE DRAWINGS

FIG. 1 is as isometric view depiction of a variation of a form suitable for use in the invention.

FIG. 2 is a depiction of the overall layers that comprise an embodiment of the device.

FIG. 3 is a depiction of a device placed against the roof of the mouth.

FIG. 4 is a depiction of a device comprised of discrete layers.

FIG. 5 is a depiction of a device comprised of continuously variable composition layers.

FIG. 6 is a depiction of a particle loaded with carbohydrate and covered with an inert time release agent layer.

FIG. 7A shows a graph of composition versus distance from one side of a device of FIG. 7B.

FIG. 8A shows a graph of the composition of the device of FIG. 8B as measured along a thickness of the device.

FIG. 9 is a cross section of a device and description of thickness of each layer.

FIG. 10A is a graph of the composition of a device of FIG. 10B as measured along a thickness of the device.

FIG. 11A is a graph of the composition of a device of FIG. 11B as measured along a thickness of the device for a continuously variable concentration of solute and inert line release agent.

FIG. 12A is a graph of the composition of a device of FIG. 12B, where the concentration is continuously variable and the device has an inert layer on an outside surface.

FIG. 13 illustrates a microparticle with solute, inert time release agent, and acid hydrolyzed layers.

FIG. 14 shows an example of a main body having a protrusion that forms a slot to accept teeth for retaining the device in the mouth.

FIG. 15 shows another example of a main body structure having a number of slots to form a resilient edge portion.

FIG. 16 shows another example of a main body having a protrusion that forms a slot to accept teeth for retaining the device in the mouth.

FIG. 17 is a depiction of a form with a plurality of protrusions adapted to retain the device within the mouth.

FIG. 18 shows a partial side view of a protrusion of the device retaining the device within the mouth by wrapping around teeth.

FIGS. 19-21 show the variations of FIGS. 14 to 17 when nested within the mouth and about the teeth.

FIG. 22 is a graph of Gut Function and the adsorption rate of glucose versus the amount of glucose ingested.

FIG. 23 is a chart of the timed release of solutes. This graph plots the theoretical change in glucose versus the time after application of the device.

FIGS. 24 to 25B show variations of additional protrusions is a depiction or a form with resilient member.

### DETAILED DESCRIPTION OF THE INVENTION

The invention includes devices and methods that provide a solute (or other substance) in a convenient manner such that the solute/substance can be delivered over a desired period of time.

In one variation, the invention includes a device for the timed release of a solute having an active agent into a stomach to increase a blood-sugar level of a human or animal. However, the principles described herein may include additional variations where the active agent is delivered for therapeutic reasons or for supplementing dietary needs. For example, additional agents for use with the device include: therapeutic agents (i.e. pharmacological agents), performance enhancement agents (creatine, steroids, etc), etc.

The timed release is made possible by the use of an inert time release agent. Typically the inert chemical or time release agent is one that provides little or no caloric value. The active agent will typically be covered by the inert time release agent or integrated with the inert time release agent. In the former case, the active solute agent will not be dissolved by saliva until the inert time release agent dissolves. Accordingly, this delay permits a timed delivery of the active solute agent as discussed herein. With regards to the latter case, the active solute agent and inert time release agent may be combined on the device as a solute layer. To effectuate a timed release, the ratio of active agent to inert time release agent will be lower towards an exposed surface of the device and higher towards the unexposed surface. Accordingly, as the initial exposed layer of time release agent dissolves, the lower ratio of the active solute agent at a surface of the device provides for a gradual dispensing of the active solute agent.

While the variations of the invention include a number of active solute agents being dispensed, for exemplary purposes, the variations discussed below primarily include active solute agents that increase a blood sugar level of the individual.

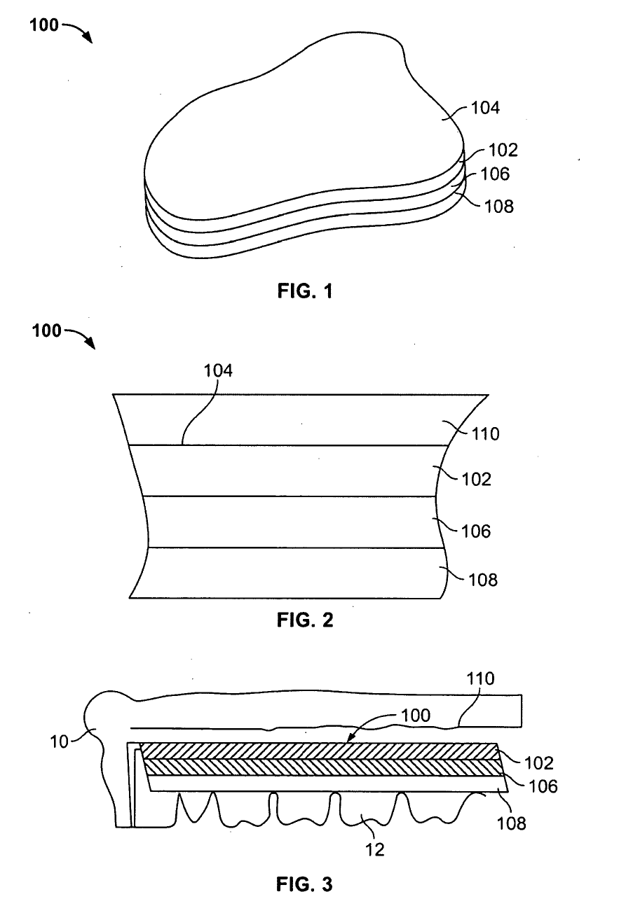

FIG. 1 shows an example of a concept of a device 100 according to the present invention. In this variation, the device includes a main body 102 having a top/engaging surface 104. The engaging surface permits removable nesting of the device 100 in a mouth of a user. In the illustrated variation, the engaging surface 104 rests against the roof of the mouth. As discussed herein, variations of the device may include engaging surfaces 104 that have an adhesive an inert time release agent or both. In addition, an adhesive, solute, or inert time release agent may be coated onto the surface 104.

The main body 102 can have any number of shapes and configurations. In addition, the main body 102 can be fabricated from a resilient polymer, a rigid polymer, or a soluble edible material. Although the shapes of the main body 102 illustrated herein fit the palate and between and/or around the teeth, variations of the main body may include any number of dental appliance type configurations that fit over the teeth such as mouth guard type configurations. Moreover, the main body may be of a shape such as a medication delivery tray. In yet additional variations, the main body may include retainer type structures that are used to align teeth, prevent snoring, and/or prevent grinding of teeth. In any case, the main body will have a surface for providing a solute layer, a pH active layer, and/or an inert time release agent layer for graduated delivery as described herein. Typically, such a surface will allow for saliva from the mouth to gradually dissolve the layer allowing for the active agent to be delivered into the digestive system.

Variations of such dental appliance configurations useful as the main body include those variations disclosed herein as well as those disclosed in U.S. Pat. Nos. 5,248,310; 6,126,443; 6,276,935 generally relating to forms of medicine delivery. U.S. Pat. Nos. 5,536,169; 5,692,894; 5,836,761; 5,580,243 generally relating to retainer type forms. U.S. Pat. Nos. 5,752,822 and 5,536,168 generally relating to mouth based snore reducing/airway devices. U.S. Pat. Nos. 5,865,619 and 5,582,517 relating to mouth guards and dental impression trays. The above patents are incorporated by reference herein.

Turning back to FIG. 1, the illustrated variation has discrete layers including an active solute layer 106 and an inert time release agent layer 108 the function of each of which is described below. Typically, the active solute layer 106 is situated so that it will encounter saliva to dissolve and release the active solute, 106. In this variation, the active solute material layer 106 is located on the main body 102 on a surface opposite to that of the engagement surface 104. However, as noted herein, variations of the device include an active layer 106 that is placed against the palate or tongue of the user.

The active solute layer 106 includes the agent that brings about the desired response in the user. Accordingly, the active solute layer 106 may also be referred to as a solute layer. In those variations of the device intended to raise blood sugar levels of the user, the solute may be dextrose, sucrose, fructose, a polysaccharide such as corn starch or other active compound that is a suitable sugar or a material that breaks down into dextrose through the digestive process.

The inert time release agent layer 108 typically covers the active solute layer 106 allowing for a gradual release of the active solute layer and material 106 into the body. This inert layer 108 slowly dissolves in saliva. In most cases, the inert time release agent layer makes no contribution to caloric content or carbohydrate content. Methylcellulose is a suitable inert time release agent layer that does not contribute any calories or food to the device. Methylcellulose may be modified by several means to create a slowly dissolving layer. One means of modification is to cross link the methylcellulose. Another method is to attach hydrophobic organic molecules such as propyl or butyl groups to the main polymer chain of the methylcellulose. A common time release agent additive is hydroxypropylmethylcellulose.

FIG. 2 shows a cross section of the device 100 of FIG. 1. As shown, the top surface 104 of the main body 102 functions as an engagement surface to retain the device 100 against a roof of the mouth of the user. Accordingly, an adhesive or other coating or layer 110 may be located on the engagement surface 104. For example, this layer 110 may contain an adhesive or be completely covered by an inert time release agent layer. The main body 102 includes an active layer 106 and an inert time release agent layer 108 as described above. It is noted that the thickness of the layer are for illustration as the amount of materials used in different variations will vary depending on the intended application.

FIG. 3 is a partial cross sectional view of a device 100 when placed in the mouth. This depiction shows the device 100 placed adjacent to the palate or the roof of the mouth and adjacent to teeth.

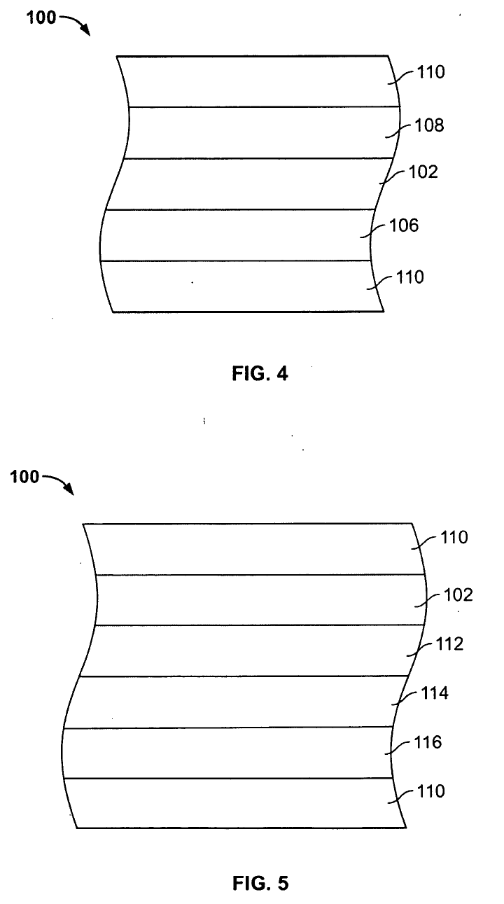

FIG. 4 represents a cross section of another variation of a device 100 having an adhesive layer 110 on a top of the device 100. In this variation, the adhesive layer 110 is placed on a layer of inert time release agent material 108 that is located on the main body 102 of the device 100. The adhesive layers described herein may be comprised of a category of oral adhesives that are based upon zinc and strontium salts in a matrix of lower alkyl vinyl ether-maleic acid copolymers. Another category of adhesive suitable for oral applications includes hydroxy-propyl-cellulose and starches. Though any oral gel or oral biocompatible adhesive would suffice. An edible adhesive is another possibility.

FIG. 5 shows another variation of a device 100 having a continuously variable layered coating. As shown, the device 100 may include a top adhesive or inert time release agent layer 110 located on a main body 102. The next layer can be an active layer 112 that has the highest concentration of active ingredient relative to the successive layers. This active layer 112 can be a solute of pure dextrose or other form of carbohydrate. The following several layers 114, 116 can be a combination of an active ingredient or a solute of carbohydrate and an inert time release agent. In this particular variation, the layers 114, 116 can have a concentration of active ingredient that varies from layer to layer. For example, the concentration of active ingredient in layer 114 may be higher than that of layer 116. The device may have any number of layers with decreasing concentrations of active ingredients until the exposed region is purely an inert time release agent 110. Such a construction provides a gradual increasing dosage of the active ingredient as the device 100 dissolves in the users mouth. As noted above, such a feature may be especially useful to maintain blood sugar levels at an acceptable range overnight.

An additional variation can include solutes such as polysaccharides such as maltose or maltodextrose, or corn starch. Polysaccharides and starches provide additional dextrose units per molecule and require additional time to break down by digestion. By loading the device with polysaccharides or starches, a cumulative or stacked bolus of dextrose is possible. The initial layers of inert time release agent may contain a polysaccharide. During digestion of the polysaccharide additional layers of inert time release agent and polysaccharide or inert time release agent and dextrose or other carbohydrate are dissolved and swallowed. Though the subsequent layers may provide immediate dextrose to the blood when swallowed, the polysaccharide from the previous layers breaks down into dextrose concurrently. By this method a greater bolus of dextrose is provided at a delayed time.

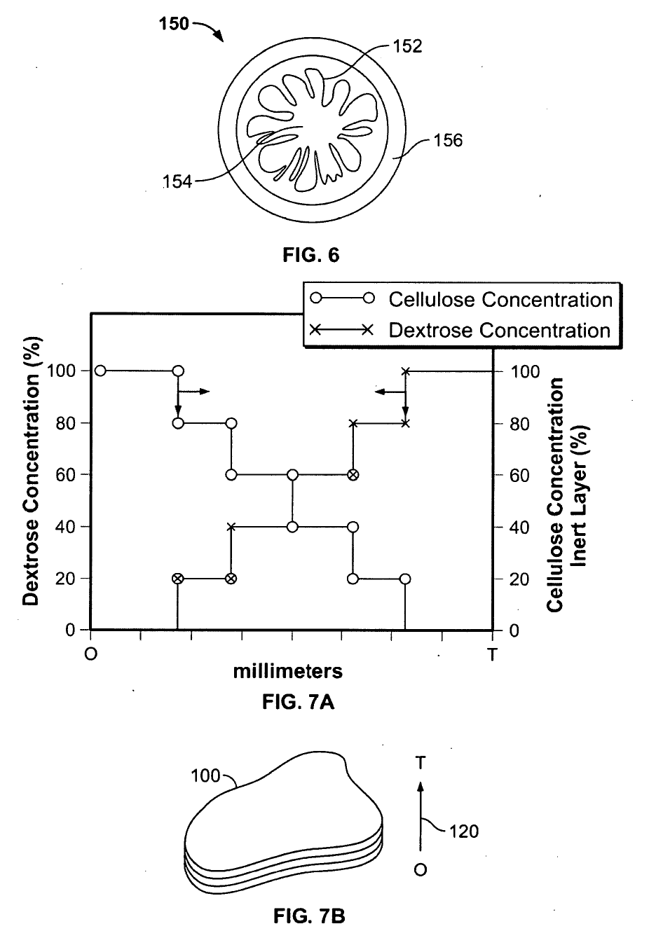

FIG. 6 shows another variation of the concepts of the invention as applied to a food substance or particle. The food particle can effectuate a gradual increase in blood sugar levels in an individual, where the particle includes a porous structure of inert time release agent material, where the inert time release agent material is gradually dissolvable in digestive fluids. The food particle also includes an active solute material located within the porous structure and adapted to increase blood sugar levels upon absorption through a digestive process, such that the gradual dissolution from and/or of the porous structure timely releases the active solute material.

The particle 154 having a number of pores or a cavity 152 includes an active solute material for a source of solute or carbohydrates. The pores 152 can provide an increase in the available surface area there by the loading of carbohydrate per unit planar surface area of the roof of the mouth. A typical time release agent particle is M5 Cabosil from the Cabot Corporation of Mattoon, Ill. The porous structure 152 can be loaded with dextrose or other suitable carbohydrate. The particles 150 can optionally be coated with an inert time release agent layer 156. The entire particle 150 with the inert time release agent layer 156 can be ingested into the stomach or the particle can bound to an inert time release agent layer and released after a finite period of time.

FIG. 7A is a chart that depicts discrete layers of solute or carbohydrate and inert time release agent, such as cellulose, as a function of distance T along a thickness 120 of the device of FIG. 7B. Typically the device 100 is affixed to the roof of a mouth where the opposite side is exposed to body fluids that deteriorate and dissolve the layers. FIG. 7A shows a percentage of cellulose concentrations and dextrose concentrations as taken along the thickness T of the device 100.

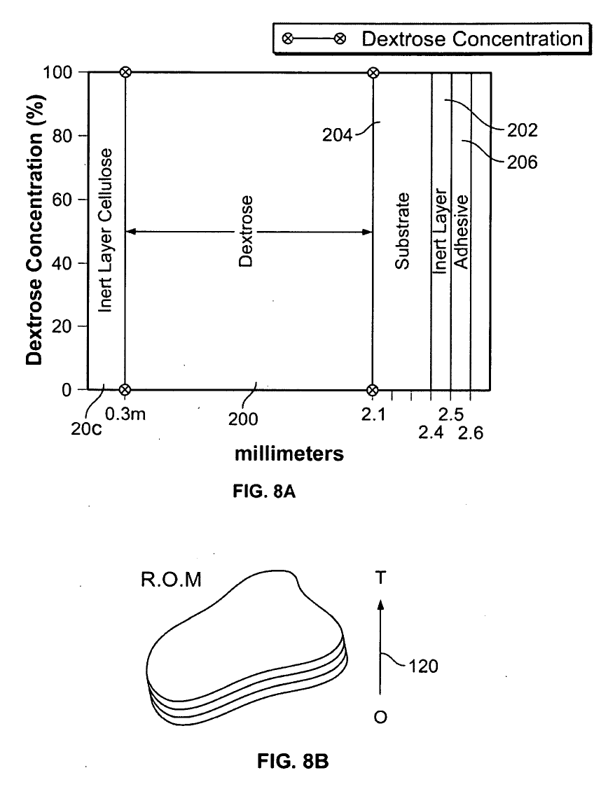

FIG. 8A is a chart depiction of the layers as a function of distance T along a thickness 120 of the device of FIG. 8B. Again, in this variation, the top surface of the device is placed against the roof of the mouth, T. FIG. 8B shows a typical loading of pure dextrose 200 under a methylcellulose layer 202 on a main body 204. Another inert layer 202 separates the main body from an adhesive layer 206. The thickness of the dextrose layer corresponds to a layer that allows the release of 4-7 grams of pure dextrose.

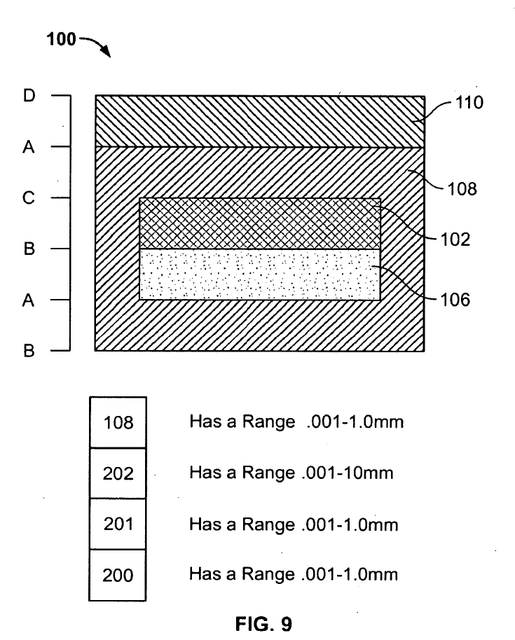

FIG. 9 shows a cross section of a device 100 that is surrounded by an inert layer 108 with an adhesive layer 110 for affixing to the mouth. The range of values for the solute corresponds to a typical loading of 4 to 10 grams of pure dextrose. For example, the thickness of the inert material 108 may range from 0.001-1.00 mm. The thickness of the active layer 106 may range from 0.001-10 mm. The thickness of the main body 102 and adhesive layer 110 may range from 0.001-1.00 mm.

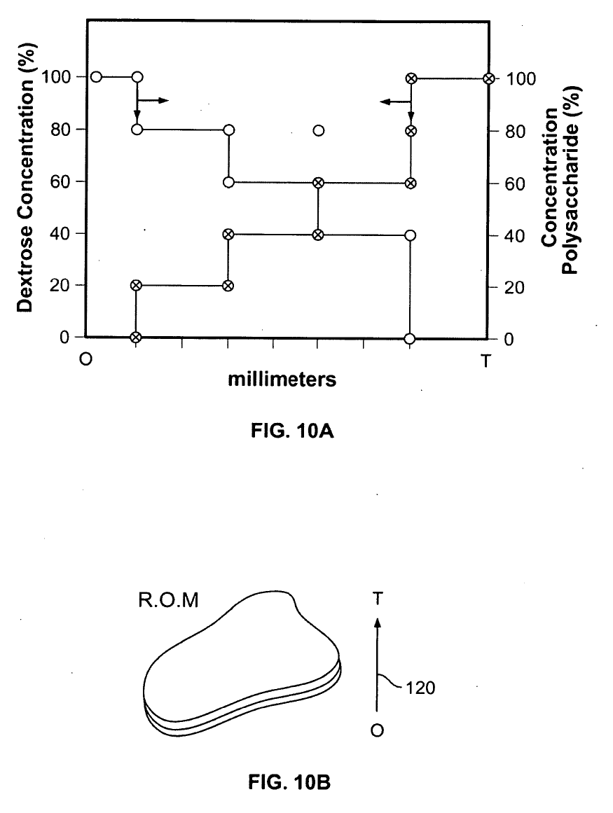

FIG. 10A shows a chart of the variation in concentration of a device that uses both a monosaccharide solute and a polysaccharide solute. As the chart shows, the concentration of the two entities discretely decreases in opposite directions from the roof of the mouth. The monosaccharide concentration increases as the distance to the roof of the mouth decreases. The polysaccharide concentration decreases as the distance to the roof of the mouth decreases. By this means the polysaccharide provides the bolus effect mentioned earlier. That is to say that the polysaccharide concentration is large farthest from the roof of the mouth. It inters the stomach first and begins to be digested. As the layers continue to be dissolved and swallowed, more and more of the monosaccharide is digested. Since the polysaccharide requires a finite amount of time to digest into a monosaccharide, such as dextrose, the combined effect is to deliver a bolus of dextrose to the blood. This provides some efficiency in loading by not wasting any of the time release layers. Each layer contains a form of carbohydrate that breaks down into or is pure dextrose.

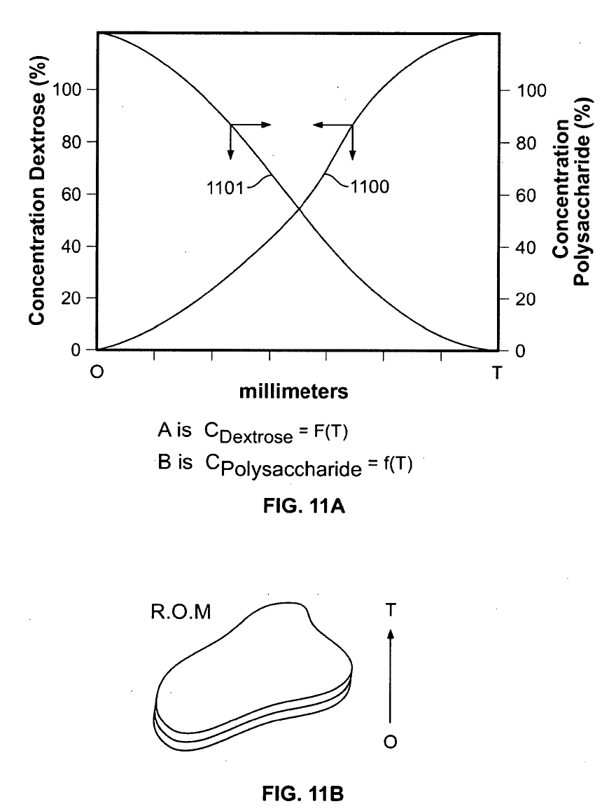

FIG. 11A depicts a continuously variable concentration of monosaccharide solute, such as dextrose, and a polysaccharide solute such as corn starch. The variation in each concentration is a function of the distance from a side of the device that is placed in contact with a roof of the mouth.

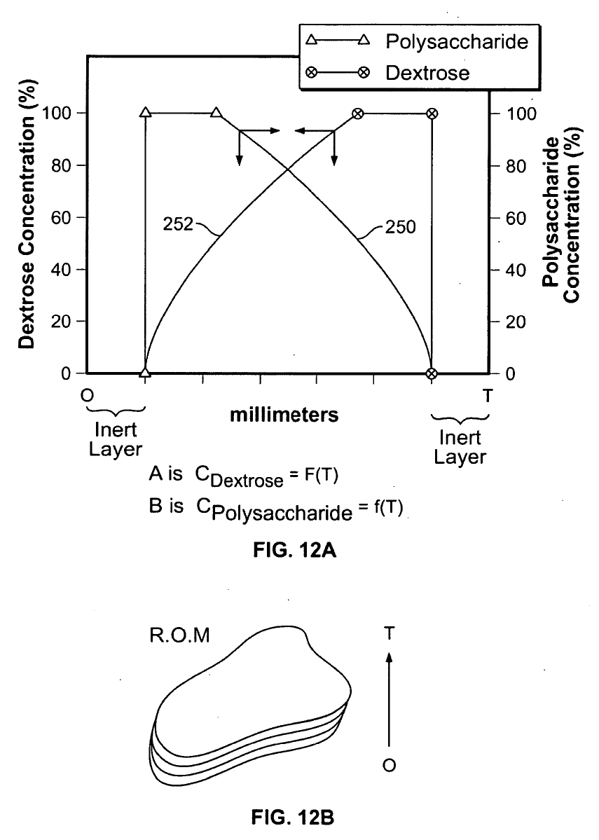

FIG. 12A shows another continuously variable concentration of monosaccharide solute, such as dextrose, and a polysaccharide solute such as corn starch. However, there is an inert time release agent layer that surrounds the carbohydrate solutes as evidenced by the end portions of the chart. In this chart curve 250 is the variation of the polysaccharide solute with distance and 252 is the variation of the monosaccharide solute with distance.

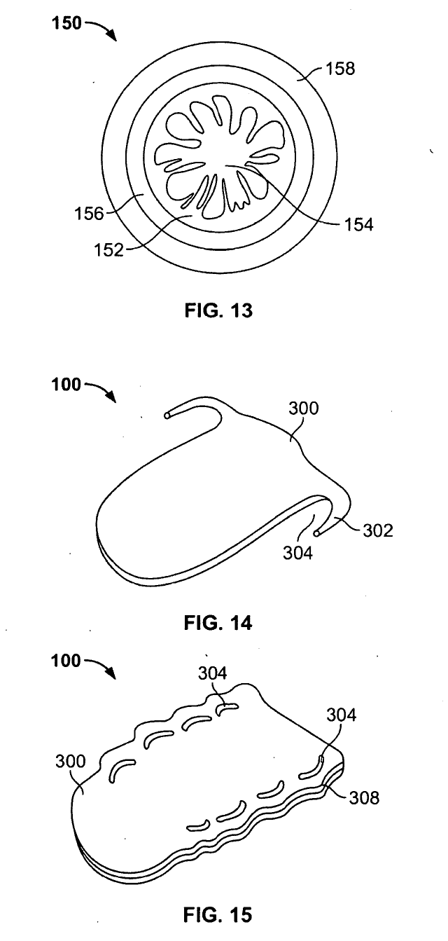

FIG. 13 depicts a particle, similar to that described in FIG. 6. However, in this variation, the particle 150 includes a pH activated layer 158. This layer 158 dissolves or breaks down in the presence of stomach acids. By coating the particle with the pH activated layer 158, the active solute material, 152, located in the pores of the particle 154 are not readily released when the particle is in the mouth. This may be advantageous to prevent dental problems such as tooth decay. The pH sensitive layer may also afford a longer time prior to release of the solute or carbohydrate. This will allow a greater length of time prior to delivery of the solute carbohydrate.

FIG. 14 shows another variation of a main body structure 300 with a protrusion 302 that assists in holding the device 100 in the mouth. In this variation, the protrusion 302 forms a slot 304. The protrusion is shaped or shapeable so that the slot 304 can either wrap around teeth or form an interference or friction fit with the teeth.

FIG. 15 is another variation of a main body structure 300 where the main body 300 includes a plurality of slots or openings 304. The device 100 uses discrete or continuous slots 304 to create a cantilever beam effect with the edge portion 308 so that the edge portion rests against the inner surface of the teeth. The edge portion 308 may be flexible or rigid. The edge portion 308 may flex to allow a form fit along the inner surface of the teeth. By this means there would be intimate contact against the teeth by the force of the edge portion 308.

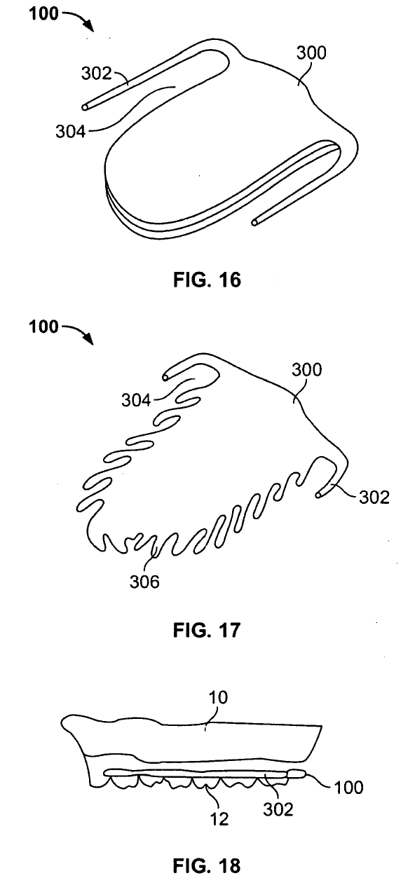

FIG. 16 shows another variation of a main body structure 300 with a protrusion 302 that assists in holding the device 100 in the mouth. In this variation, the protrusion 302 extends to create a longer slot 304 that is intended to wrap around several teeth.

FIG. 17 shows a variation of a device 100 having multiple protrusions 302 306 where the first protrusions 302 define a slot for nesting about teeth. The second protrusions 306 act as resilient members that apply force against the inside and outside of the teeth.

FIG. 18 shows a partial side view of the device with a protrusion 304 placed in the mouth. The protrusion 304 applies a resilient force to the teeth and holds the device 100 in place.

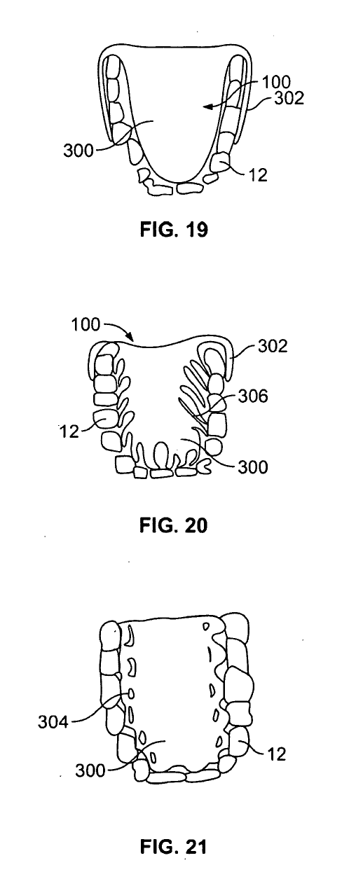

FIGS. 19 to 21 show the variations of FIGS. 14 to 17 discussed above when nested within the mouth and about the teeth.

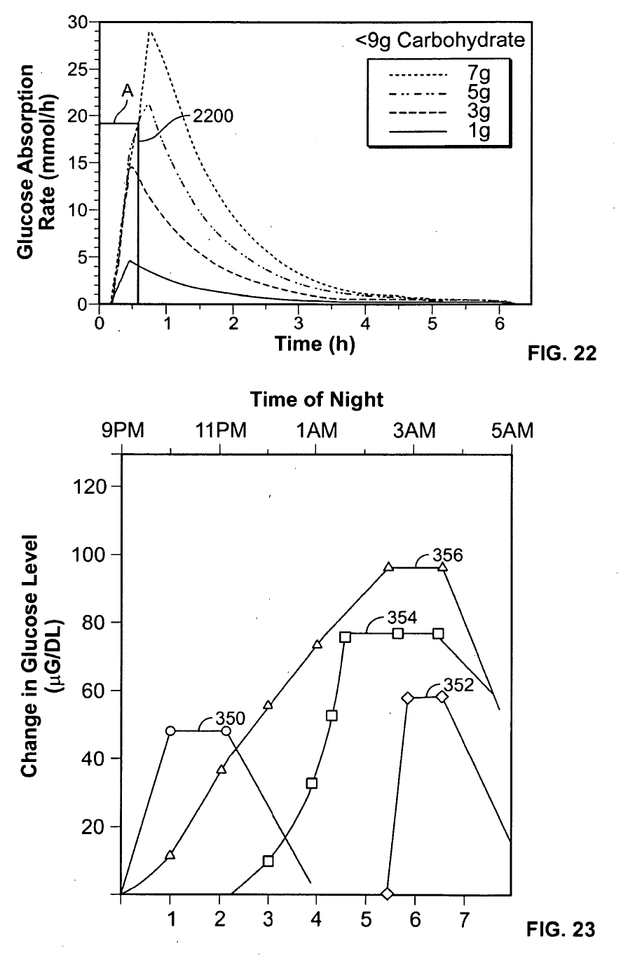

FIG. 22 is publicly available information representing a graph of the glucose adsorption rate versus time for various boluses of glucose when ingested via the conventional means of taking the substances. This curve demonstrates that a 4 to 7 gram quantity of glucose will raise the blood sugar level by 3.3 mMolar. This is equivalent to 60 mg/dl. The dose calculation is based upon a human blood volume of 5.6 liters. This represents 18.67 mMoles per human being. On the graph at 18.67 mMoles, item 2200, corresponds to a dose between 4 and 7 grams. Accordingly, such information may be used to determine the dosage required by the particular needs of the patient.

FIG. 23 is a graphical depiction of the time released behavior, namely the change in glucose level as a function of time. This data for this curve 350 is taken from the Glucerna, product package. The three additional curves 352, 354, 356 represent the theoretical, proposed behavior of select, though not limiting, embodiments of the invention herein. They are for dextrose+cellulose 352, polysaccharide+dextrose 354 and polysaccharide+dextrose+cellulose 356.

When these substances are applied for use in the invention, it is estimated that the time release behavior should be similar. As shown on the curve the entire effect of ingesting a Glucerna bar is gone by 2:00 AM if the patient eats the bar between 9:00 and 10:00 PM. The proposed, theoretical curve 352 is for a dextrose and inert time release agent layer, cellulose. This shows that the inert lime release agent layer prevents contact of the dextrose with saliva until 2:00 AM. At that time the dextrose enters the stomach and is adsorbed into the blood stream 30 to 60 minutes later. A 5 gram dose of glucose will boost the blood sugar level by 60 mg/dl for an hour and keep the blood sugar elevated for up to 2 hours. A 7 gram dose of glucose will keep the blood sugar elevated for 2.5 hours. The curve 356 is the proposed, theoretical effect of a polysaccharide solute plus a monosaccharide solute, such as dextrose and minimal if any inert time release agent. The first layer to dissolve and enter the stomach is the polysaccharide. This carbohydrate has a delayed, but amplifying effect since each molecule delivers several dextrose units. The polysaccharide is digested over time, typically 1 to 2 hours into dextrose. Concurrently, the dextrose layers are exposed and a bolus of dextrose is delivered to the blood stream. The curve 356 is a proposed, theoretical combination of cellulose, polysaccharide, and monosaccharide. This curve has the delayed effect due to the inert time release agent layer, cellulose and a subtle amplification effect by the polysaccharide. The mixture of the polysaccharide and the monosaccharide provide an increase in blood sugar over a longer period of time than pure dextrose.

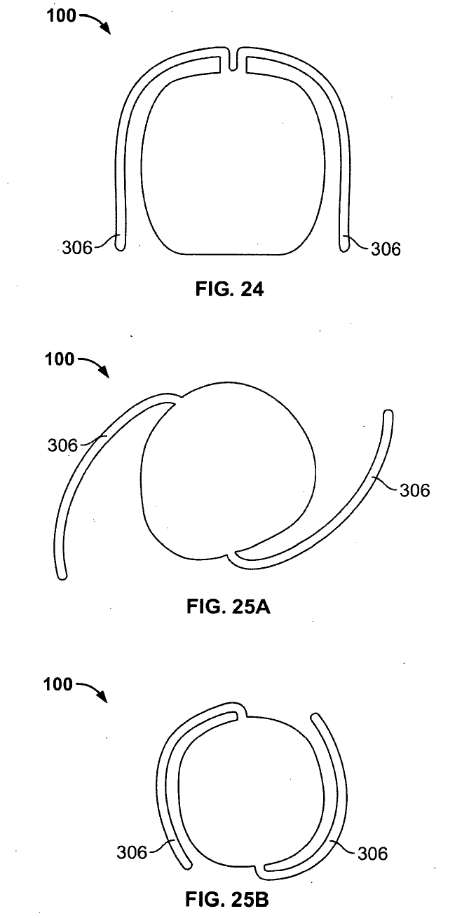

FIG. 24 and FIGS. 25A and 25B show additional variations of protrusions 306 that that press against the inside of the teeth. These resilient protrusions 306 may be collapsed with a fixture or component of the system that allows ease of placement. When the fixture is removed the resilient member expands outward against the teeth. FIG. 25A represents a spiral resilient member that may allow the device to be placed more independent of orientation in the mouth.

1. An oral appliance for retention within a mouth and effectuating a gradual increase in blood sugar levels, the appliance comprising:
a main body having a carrier surface and a engaging surface, where the engaging surface permits removable nesting and retaining of the main body within the mouth; and
an active solute material located on the carrier surface, where the active solute material increases blood sugar levels upon absorption through a digestive process.

2. The oral appliance of claim 1, further comprising an inert time release agent layer being soluble in saliva and covering the active solute material, such that break down of the inert time release agent layer causes release of the active solute material into the mouth.

3. The oral appliance of claim 2, where the inert time release agent layer comprises methylcellulose.

4. The oral appliance of claim 2, where the inert time release agent layer is cross-linked to slow a dissolving rate of the inert time release agent layer.

5. The oral appliance of claim 2, where the inert time release agent layer comprises a layer of hydrophobic organic molecules.

6. The oral appliance of claim 2, where the inert time release agent layer comprises M5 Cabosil.

7. The oral appliance of claim 1, further comprising an adhesive material on the engaging surface.

8.-10. (canceled)

11. The oral appliance of claim 1, where the main body further comprises at least one protrusion defining a slot adjacent to the main body, where the engaging surface comprise a surface of the slot and where the slot forms an interference fit with at least one tooth in the mouth.

12. The oral appliance of claim 1, where the main body further comprises a plurality of protrusions extending from at least a portion of a perimeter of the main body, where the plurality of protrusions apply a force against an interior surface of at least two teeth to retain the main body within the mouth.

13. (canceled)

14. The oral appliance of claim 1, where the main body further includes a plurality of slits defining a cantilevered portion, where the engagement surface comprises an exterior of the cantilevered portion.

15. The oral appliance of claim 1, further comprising a second inert time release agent layer over the engaging surface.

16. The oral appliance of claim 1, where the active solute material comprises a substance selected from a group consisting of glucose, maltose, maltodextrose, corn starch, dextrose, sucrose, fructose, polysaccharide, monosaccharide, a sugar, fats, protein, and a carbohydrate.

17.-19. (canceled)

20. The oral appliance of claim 1, where the active solute material is further coated with a pH activated layer.

21. (canceled)

22. The oral appliance of claim 1, where the active solute material and or the inert time release agent are coated with a pH activated layer.

23.-25. (canceled)

26. The oral appliance of claim 1, where an amount of the active solute material is sufficient to allow absorption of at least 4 grams of dextrose.

27. (canceled)

28. The oral appliance of claim 1, further comprising an additive selected from the group comprising a flavor additive, a food stabilizer, a preservative, and a food coloring dye.

29. The oral appliance of claim 1, where the main body comprises a digestible material.

30. (canceled)

31. The oral appliance of claim 1, where the main body comprises an ingestible material that is non-digestible.

32.-33. (canceled)

34. The oral appliance of claim 1, where the main body has a shape selected from a group consisting of: a retainer, a mouth guard, and a dental tray,

35. An oral appliance for retention within a mouth and effectuating a gradual increase in blood sugar levels, the appliance comprising:
a main body having a surface for nesting and retaining within the mouth; and
a layer of soluble material located on the main body, where the soluble material comprises an active solute material adapted to increase blood sugar levels upon absorption through a digestive process and an inert time release agent material, where a ratio of the active solute material to inert time release agent material varies along a depth of the layer.

36. The oral appliance of claim 35, where at an exposed surface of the layer of soluble material, the ratio of active solute material to inert time release agent material is lowest such that saliva initially dissolves a greater amount of inert time release agent material before active solute material.

37. The oral appliance of claim 36, where the ratio of active solute material to inert time release agent material increases in a direction towards the main body of the appliance.

38. The oral appliance of claim 35, further comprising an additional layer of inert time release agent on the soluble layer.

39.-45. (canceled)

46. The oral appliance of claim 35, where the main body further comprises at least one protrusion defining a slot adjacent to the main body, where the slot forms an interference fit with at least one tooth in the mouth.

47. The oral appliance of claim 35, where the main body further comprises a plurality of protrusions extending from at least a portion of a perimeter of the main body, where the plurality of protrusions apply a force against an interior surface of at least two teeth to retain the main body within the mouth.

48. The oral appliance of claim 35, where a perimeter of the main body is sized to fit within the mouth.

49. The oral appliance of claim 35, where the main body further includes a plurality of slits defining a cantilevered portion, where deformation of the cantilevered portion against teeth permits retaining of the main body in the mouth.

50. The oral appliance of claim 35, where the active solute material comprises a substance selected from a group consisting of glucose, maltose, maltodextrose, corn starch, dextrose, sucrose, fructose, polysaccharide, monosaccharide, a sugar, fats, protein, and a carbohydrate.

51. The oral appliance of claim 35, where the active solute material and or the inert time release agent is further coated with a pH activated layer.

52.-54. (canceled)

55. The oral appliance of claim 35, where an amount of the active solute material is sufficient to allow absorption of at least 2 grams of dextrose.

56. The oral appliance of claim 55, where an amount of the active solute material is sufficient to allow absorption of no more than 100 grams of dextrose.

57. (canceled)

58. The oral appliance of claim 35, where the main body comprises a digestible material.

59. (canceled)

60. The oral appliance of claim 35, where the main body comprises an ingestible material that is non-digestible.

61.-62. (canceled)

63. The oral appliance of claim 35, where the main body has a shape selected from a group consisting of: a retainer, a mouth guard, and a dental tray,

64. A particle for effecting a gradual increase in blood sugar levels in an individual, the particle comprising:
a porous structure comprising an inert time release agent material, where the inert time release agent material is gradually dissolvable in digestive fluids; and
an active solute material located within the porous structure and adapted to increase blood sugar levels upon absorption through a digestive process, such that the gradual dissolving of the porous structure timely releases the active solute material.

65. The particle of claim 64, where the porous structure is coated with an additional layer of active solute material.

66. The particle of claim 64, where the porous structure is coated with an additional layer of inert time release agent material.

67. The particle of claim 64, where a ratio of active solute material to inert time release agent material is greatest towards a center of the porous structure.

68. The particle of claim 67, where a ratio of active solute material to inert time release agent material increases in a direction towards the main body of the appliance.

69. (canceled)

70. The particle of claim 64, where the inert time release agent material is cross-linked to slow a dissolving rate of the inert time release agent layer.

71. (canceled)

72. The particle of claim 64, where the active solute material and or the inert time release material is further coated with a pH activated layer.

73.-78. (canceled)

79. The particle of claim 64, where the main body comprises a digestible material.

80. (canceled)

81. The particle of claim 64, where the main body comprises an ingestible material that is non-digestible.

82.-83. (canceled)

84. An oral appliance for retention within a mouth and effectuating a gradual release of a substance, the appliance comprising:
a main body having a surface for nesting and retaining within the mouth; and
a layer of soluble material located on the main body, where the soluble material comprises an active solute material adapted to effectuate a change in a body through a digestive process and an inert time release agent material, where a ratio of the active solute material to inert time release agent material varies along a depth of the layer.

85. The oral appliance of claim 84, where the active solute material comprises therapeutic agents.

86. The oral appliance of claim 85, where the therapeutic agents pharmaceutical or pharmacological agents.

87. The oral appliance of claim 84, where the active solute material comprises performance enhancing agents.

88. The oral appliance of claim 87, where the active solute material comprises a substance selected from a group consisting of creatine and steroids.

89. The oral appliance of claim 84, where the main body comprises a digestible material.

90.-91. (canceled)
